(19) United States
(12) Patent Application Publication
(10) Pub. No.: US 2013/0178503 A1
(43) Pub. Date: Jul. 11, 2013

(54) METHODS OF TREATING HEMATOLOGIC MALIGNANCIES USING 6-CYCLOHEXYL-1-HYDROXY-4-METHYL-2(1H)-PYRIDONE

(71) Applicants: Frank Mercurio, Rancho Santa Fe, CA (US); Kyle W.H. Chan, San Diego, CA (US)

(72) Inventors: Frank Mercurio, Rancho Santa Fe, CA (US); Kyle W.H. Chan, San Diego, CA (US)

(73) Assignee: BIOTHERYX INC., San Diego, CA (US)

(21) Appl. No.: 13/701,486

(22) PCT Filed: Jun. 1, 2011
(86) PCT No.: PCT/US2011/038702

§ 371 Date: Mar. 29, 2013

Related U.S. Application Data
(60) Provisional application No. 61/350,438, filed on Jun. 1, 2010.

(51) Int. Cl. A61K 31/4418

(52) U.S. Cl. CPC A61K 31/4418
USPC 514/345; 435/375

(57) ABSTRACT

Provided herein are methods of treating a drug-resistant hematologic malignancy in a subject, which comprises administering to the subject a therapeutically effective amount of 6-cyclohexyl-1-hydroxy-4-methyl-2(1H)-pyridone, or a pharmaceutical salt or solvate thereof.

### CROSS REFERENCE TO RELATED APPLICATION

This application claims priority to U.S. Provisional Application No. 61/350,438, filed Jun. 1, 2010, the disclosure of which is incorporated herein by reference in its entirety.

### FIELD

Provided herein are methods of treating a drug-resistant hematologic malignancy in a subject, which comprises administering to the subject a therapeutically effective amount of 6-cyclohexyl-1-hydroxy-4-methyl-2(1H)-pyridone, or a pharmaceutical salt or solvate thereof.

### BACKGROUND

Hematologic or hematopoietic malignancies are cancers of the blood or bone marrow, including leukemia and lymphoma. Leukemia is characterized by the uncontrolled accumulation of blood cells, which is categorized into four types: acute lymphocytic leukemia (ALL), acute myelogenous leukemia (AML), chronic lymphocytic leukemia (CLL), and chronic myelogenous leukemia (CML). Acute leukemia is a rapidly progressing disease that results in the accumulation of immature, functionless cells in the marrow and blood. The marrow often stops producing enough normal red cells, white cells and platelets. On the other hand, chronic leukemia progresses more slowly and allows greater numbers of more mature, functional cells to be made. Chronic leukemias account for 11 percent more cases than acute leukemias.

It was estimated that 245,225 people in the United States were living with, or were in remission from, leukemia in 2009. Leukemia was expected to strike more than 10 times as many adults as children in 2009 (About 44,790 adults compared with 3,509 children, aged 0-14 years). The most common types of leukemia in adults are acute myelogenous leukemia (AML), with estimated 12,810 new cases in 2009, and chronic lymphocytic leukemia (CLL), with about 15,490 new cases in 2009. Chronic myelogenous leukemia (CML) was estimated to affect about 5,050 persons in 2009. The most common type of leukemia in children is acute lymphocytic leukemia (ALL), which was estimated to affect about 5,760 persons in 2009.

While current chemotherapy can result in complete remissions, the long term disease-free survival rate for leukemias, in particular AML, is low. For example, the survival rate for AML was estimated to be less than about 20% in 2009. Therefore, there is a clear and unmet need for effective therapeutics for treatment of hematologic malignancies, including leukemias.

### SUMMARY OF THE DISCLOSURE

Provided herein are methods of treating a drug-resistant hematologic malignancy in a subject, which comprises administering to the subject a therapeutically effective amount of 6-cyclohexyl-1-hydroxy-4-methyl-2(1H)-pyridone, or a pharmaceutical salt or solvate thereof. In certain embodiments, the drug-resistant hematologic malignancy is not imatinib-resistant CML.

In one embodiment, the drug-resistant hematologic malignancy is drug-resistant leukemia, with the proviso that the leukemia is not imatinib-resistant CML.

In another embodiment, the leukemia is resistant to a Bcr-Abl kinase inhibitor, with the proviso that the leukemia is not imatinib-resistant CML. In certain embodiments, the leukemia is resistant to imatinib, dasatinib, nilatinib, or bosutinib, with the proviso that the leukemia is not imatinib-resistant CML. In certain embodiments, the leukemia is resistant to cytarabine. In certain embodiments, the leukemia is resistant to vincristine. In certain embodiments, the drug-resistant leukemia is Philadelphia positive. In certain embodiments, the drug-resistant leukemia is relapsed or refractory.

In yet another embodiment, the leukemia is drug-resistant acute leukemia. In certain embodiments, the acute leukemia is resistant to a Bcr-Abl kinase inhibitor. In certain embodiments, the acute leukemia is resistant to imatinib, dasatinib, nilatinib, or bosutinib. In certain embodiments, the acute leukemia is resistant to cytarabine. In certain embodiments, the acute leukemia is resistant to vincristine. In certain embodiments, the drug-resistant acute leukemia is Philadelphia positive. In certain embodiments, the drug-resistant acute leukemia is relapsed or refractory.

In yet another embodiment, the drug-resistant acute leukemia is drug-resistant ALL. In certain embodiments, the ALL is resistant to a Bcr-Abl kinase inhibitor. In certain embodiments, the ALL is resistant to imatinib, dasatinib, nilatinib, or bosutinib. In certain embodiments, the ALL is resistant to cytarabine. In certain embodiments, the ALL is resistant to vincristine. In certain embodiments, the drug-resistant ALL is Philadelphia positive. In certain embodiments, the drug-resistant ALL is relapsed or refractory.

In yet another embodiment, the drug-resistant acute leukemia is drug-resistant AML. In certain embodiments, the AML is resistant to a Bcr-Abl kinase inhibitor. In certain embodiments, the AML is resistant to imatinib, dasatinib, nilatinib, or bosutinib. In certain embodiments, the AML is resistant to cytarabine. In certain embodiments, the AML is resistant to vincristine. In certain embodiments, the drug-resistant AML is Philadelphia positive. In certain embodiments, the drug-resistant AML is relapsed or refractory.

In yet another embodiment, the drug-resistant leukemia is drug-resistant chronic leukemia, with the proviso that the leukemia is not imatinib-resistant CML. In certain embodiments, the chronic leukemia is resistant to a Bcr-Abl kinase inhibitor with the proviso that the chronic leukemia is not imatinib-resistant CML. In certain embodiments, the chronic leukemia is resistant to imatinib, dasatinib, nilatinib, or bosutinib, with the proviso that the chronic leukemia is not imatinib-resistant CML. In certain embodiments, the chronic leukemia is resistant to cytarabine. In certain embodiments, the chronic leukemia is resistant to vincristine. In certain embodiments, the drug-resistant chronic leukemia is Philadelphia positive. In certain embodiments, the drug-resistant chronic leukemia is relapsed or refractory.

In yet another embodiment, the drug-resistant chronic leukemia is drug-resistant CLL. In certain embodiments, the CLL is resistant to a Bcr-Abl kinase inhibitor. In certain embodiments, the CLL is resistant to imatinib, dasatinib, nilatinib, or bosutinib. In certain embodiments, the CLL is resistant to cytarabine. In certain embodiments, the CLL is resistant to vincristine. In certain embodiments, the drug-resistant CLL is Philadelphia positive. In certain embodiments, the drug-resistant CLL is relapsed or refractory.

In still another embodiment, the drug-resistant chronic leukemia is drug-resistant CML, with the proviso that the CML is not imatinib-resistant. In certain embodiments, the CML is resistant to a Bcr-Abl kinase inhibitor, with the proviso that the CML is not imatinib-resistant. In certain embodiments, the CML is resistant to dasatinib, nilatinib, or bosutinib. In certain embodiments, the CML is resistant to cytarabine. In certain embodiments, the CML is resistant to vincristine. In certain embodiments, the drug-resistant CML is Philadelphia positive. In certain embodiments, the drug-resistant CML is relapsed or refractory.

Also provided herein is a method for treating leukemia in a subject, which comprises oral administration of 6-cyclohexyl-1-hydroxy-4-methyl-2(1H)-pyridone, or a pharmaceutical salt or solvate thereof, to the subject at a dosage of about 2, about 5, about 10, about 15, or about 20 mg/kg/day. In one embodiment, the leukemia is drug resistant.

Further provided herein is a method for treating leukemia in a subject, which comprises intravenous administration of 6-cyclohexyl-1-hydroxy-4-methyl-2(1H)-pyridone, or a pharmaceutical salt or solvate thereof, to the subject at a dosage ranging from about 0.01 to about 10 mg/kg/day. In one embodiment, the leukemia is drug resistant.

Provided herein is a method for treating leukemia in a subject, which comprises administration of 6-cyclohexyl-1-hydroxy-4-methyl-2(1H)-pyridone, or a pharmaceutical salt or solvate thereof, in an amount sufficient to provide a plasma concentration of 6-cyclohexyl-1-hydroxy-4-methyl-2(1H)-pyridone at steady state ranging from about 1 to about 20 μM. In one embodiment, the leukemia is drug resistant.

Provided herein is a method for treating leukemia in a subject, which comprises administration of 6-cyclohexyl-1-hydroxy-4-methyl-2(1H)-pyridone, or a pharmaceutical salt or solvate thereof, in an amount sufficient to provide a maximum plasma concentration of 6-cyclohexyl-1-hydroxy-4-methyl-2(1H)-pyridone ranging from about 1 to about 50 μM. In one embodiment, the leukemia is drug resistant.

Provided herein is a method for treating leukemia in a subject, which comprises administration of 6-cyclohexyl-1-hydroxy-4-methyl-2(1H)-pyridone, or a pharmaceutical salt or solvate thereof, in an amount sufficient to provide a maximum plasma concentration of 6-cyclohexyl-1-hydroxy-4-methyl-2(1H)-pyridone ranging from about 1 to about 50 μM when two or more doses of 6-cyclohexyl-1-hydroxy-4-methyl-2(1H)-pyridone are administered. In one embodiment, the leukemia is drug resistant.

Provided herein is a method of inhibiting the growth of a leukemia stem cell, comprising the step of contacting the cell with 6-cyclohexyl-1-hydroxy-4-methyl-2(1H)-pyridone, or a pharmaceutical salt or solvate thereof. In one embodiment, the leukemia stem cell is drug resistant.

### BRIEF DESCRIPTION OF THE DRAWINGS

FIG. 1 shows effects of cytarabine on KG-1a cancer cell lines after 72-hour treatment, where cytarabine has an IC₅₀ value of about 0.625 μM against cytarabine-nonresistant KG-1a and about 20 μM against cytarabine-resistant KG-1aCR.

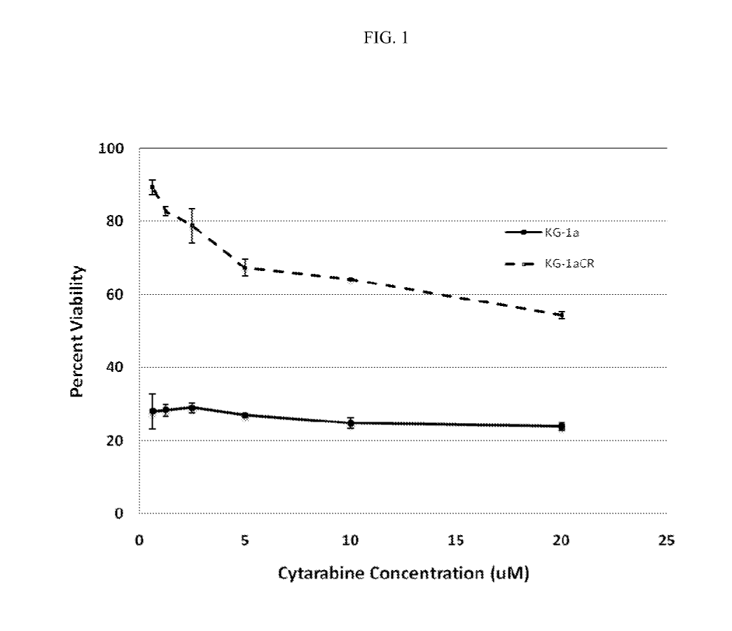

FIG. 2 shows effects of 6-cyclohexyl-1-hydroxy-4-methyl-2(1H)-pyridone (CPX) on KG-1a cancer cell lines after 72-hour treatment, where CPX has an IC₅₀ value of 2 μM against cytarabine-nonresistant KG-1a and about 6 μM against cytarabine-resistant KG-1 aCR.

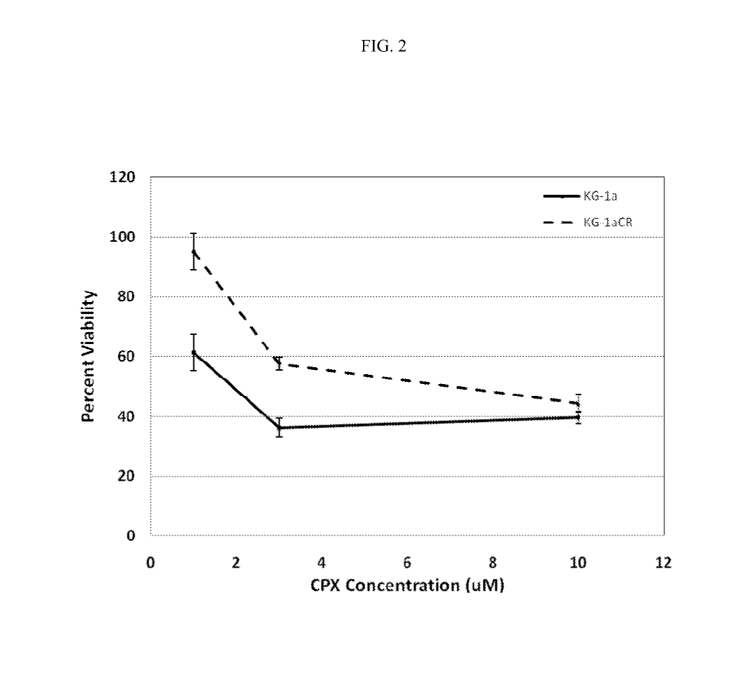

FIG. 3 shows effects of 6-cyclohexyl-1-hydroxy-4-methyl-2(1H)-pyridone (CPX) on Nalm-6 cancer cell lines after 72-hour treatment, where Nalm-6 VR is vincristine resistant cell line, where vincristine was determined to have an IC₅₀ value of about 1 nM against vincristine-nonresistant Nalm-6 and about 32 nM against vincristine-resistant Nalm-6 VR.

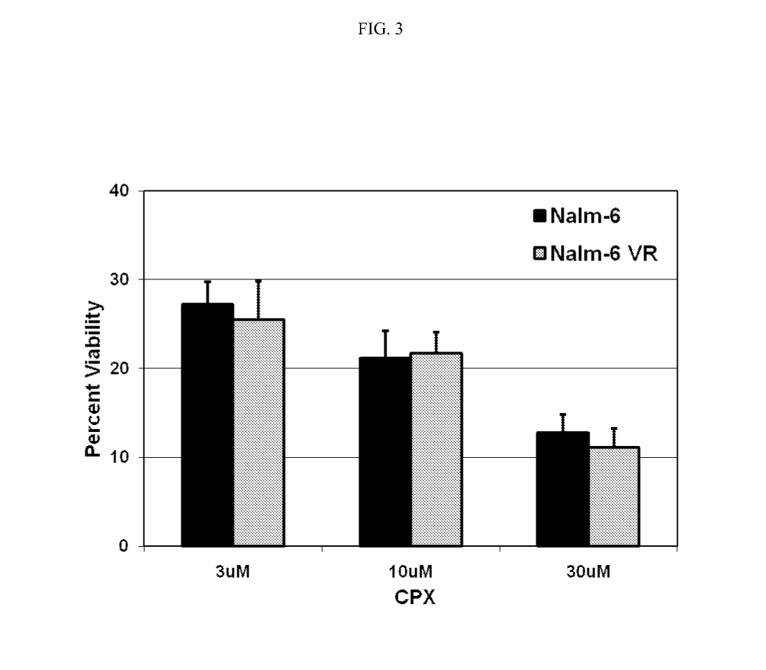

### DETAILED DESCRIPTION

To facilitate understanding of the disclosure set forth herein, a number of terms are defined below.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate

(e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The term “hematologic malignancy” refers to cancer of the body's blood-forming and immune system—the bone marrow and lymphatic tissue. Examples of hematological malignancies include, for instance, myelodysplasia, lymphomas, leukemias, lymphomas (non-Hodgkin's lymphoma), Hodgkin's disease (also called Hodgkin's lymphoma), and myeloma, such as acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocyctic leukemia (JMML), adult T-cell ALL, AML with trilineage myelodysplasia (AML/TMDS), mixed lineage leukemia (MLL), myelodysplastic syndromes (MDSs), myeloproliferative disorders (MPD), and multiple myeloma (MM).

The term “leukemia” refers to malignant neoplasms of the blood-forming tissues, including, but not limited to, chronic lymphocytic leukemia, chronic myelocytic leukemia, acute lymphoblastic leukemia, acute myeloid leukemia and acute myeloblastic leukemia. The leukemia can be relapsed, refractory, or resistant to conventional therapy.

The term “relapsed” refers to a situation where a subject or a mammal, who has had a remission of cancer after therapy has a return of cancer cells.

The term “refractory or resistant” refers to a circumstance where a subject or a mammal, even after intensive treatment, has residual cancer cells in his body.

The term “drug resistance” refers to the condition when a disease does not respond to the treatment of a drug or drugs. Drug resistance can be either intrinsic, which means the disease has never been responsive to the drug or drugs, or it can be acquired, which means the disease ceases responding to a drug or drugs that the disease had previously responded to. In certain embodiments, drug resistance is intrinsic. In certain embodiments, the drug resistance is acquired.

### Compounds

The compound suitable for use in the methods provided herein is 6-cyclohexyl-1-hydroxy-4-methyl-2(1H)-pyridone, or a pharmaceutically acceptable salt or solvate thereof, which is also known as ciclopirox and has the structure of:

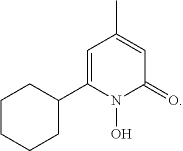

Ciclopirox is commercially available. Ciclopirox can also be prepared, isolated, or obtained by any method known to one of skill in the art. For an example, ciclopirox can be prepared according to the methods described in U.S. Pat. Nos. 3,883,545 and 3,972,888, the disclosure of each of which is incorporated herein by reference in its entirety.

In one embodiment, the ciclopirox used in the methods provided herein is a free base. In one embodiment, the free base is a solid. In another embodiment, the free base is a solid in an amorphous form. In yet another embodiment, the free base is a solid in a crystalline form.

In another embodiment, the ciclopirox used in the methods provided herein is a pharmaceutically acceptable solvate of the free base. In one embodiment, the solvate is a hydrate.

In yet another embodiment, the ciclopirox used in the methods provided herein is a pharmaceutically acceptable salt, which includes, but is not limited to, magnesium hydroxide, calcium hydroxide, potassium hydroxide, zinc hydroxide, sodium hydroxide, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

In certain embodiments, the pharmaceutically acceptable salt is an inorganic salt of ciclopirox. In certain embodiments, the pharmaceutically acceptable salt is an organic salt of ciclopirox. In certain embodiments, the pharmaceutically acceptable salt is a primary amine salt of ciclopirox. In certain embodiments, the pharmaceutically acceptable salt is a secondary amine salt of ciclopirox. In certain embodiments, the pharmaceutically acceptable salt is a tertiary amine salt of ciclopirox. In certain embodiments, the pharmaceutically acceptable salt is a quaternary amine salt of ciclopirox. In certain embodiments, the pharmaceutically acceptable salt is an aliphatic amine salt of ciclopirox. In certain embodiments, the pharmaceutically acceptable salt is an aromatic amine salt of ciclopirox. In certain embodiments, the pharmaceutically acceptable salt is ciclopirox ethanolammonium.

### Pharmaceutical Compositions

In one embodiment, provided herein are pharmaceutical compositions comprising ciclopirox, or a pharmaceutically acceptable salt or solvate thereof; in combination with a pharmaceutically acceptable vehicle, carrier, diluent, excipient, or a mixture thereof.

The pharmaceutical compositions that comprise ciclopirox can be formulated in various dosage forms for oral, parenteral, and topical administration. The pharmaceutical compositions can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, 2nd Edition, Rathbone et al., Eds., Marcel Dekker, Inc.: New York, N.Y., 2008).

In one embodiment, the pharmaceutical compositions provided herein are formulated in a dosage form for oral administration, which comprise ciclopirox, or a pharmaceutically acceptable salt or solvate thereof; and one or more pharmaceutically acceptable excipients or carriers.

In another embodiment, the pharmaceutical compositions provided herein are formulated as a suspension for oral administration, which comprise ciclopirox, or a pharmaceutically acceptable salt or solvate thereof; and one or more pharmaceutically acceptable excipients or carriers. In one embodiment, the suspension provided herein comprises ciclopirox ethanolamine salt, and two or more excipients or carriers selected from the group consisting of water, glycerin, sorbitol, sodium saccharin, xanthan gum, flavoring, citric acid, sodium citrate, methylparaben, propylparaben, and potassium sorbate. In another embodiment, the suspension provided herein comprises ciclopirox ethanolamine salt, and water, glycerin, sorbitol, sodium saccharin, xanthan gum, flavoring, citric acid, sodium citrate, methylparaben, propylparaben, and potassium sorbate. In yet another embodiment, the suspension provided herein contains 100 mg/L ciclopirox in a solution consisting of water, glycerin, sorbitol, sodium saccharin, xanthan gum, flavoring, citric acid, sodium citrate, methylparaben, propylparaben, and potassium sorbate.

In yet another embodiment, the pharmaceutical compositions provided herein are formulated in a dosage form for parenteral administration, which comprise ciclopirox, or a pharmaceutically acceptable salt or solvate thereof; and one or more pharmaceutically acceptable excipients or carriers. In one embodiment, the pharmaceutical compositions provided herein are formulated in a dosage form for intravenous administration. In another embodiment, the pharmaceutical compositions provided herein are formulated in a dosage form for subcutaneous administration. In yet another embodiment, the pharmaceutical compositions provided herein are formulated in a dosage form for intramuscular administration.

In yet another embodiment, the pharmaceutical compositions provided herein are formulated in a dosage form for topical administration, which comprise ciclopirox, or a pharmaceutically acceptable salt or solvate thereof; and one or more pharmaceutically acceptable excipients or carriers.

In yet another embodiment, the pharmaceutical compositions provided herein are formulated as a cream for topical administration, which comprise ciclopirox, or a pharmaceutically acceptable salt or solvate thereof; and one or more pharmaceutically acceptable excipients or carriers. In one embodiment, the cream provided herein comprises ciclopirox ethanolamine salt, and two or more excipients or carriers selected from the group consisting of water, octyldodecanol, mineral oil, stearyl alcohol, cocamide DEA, polysorbate 60, myristyl alcohol, sorbitan monostearate, lactic acid, and benzyl alcohol. In another embodiment, the cream provided herein comprises ciclopirox ethanolamine salt, and water, octyldodecanol, mineral oil, stearyl alcohol, cocamide DEA, polysorbate 60, myristyl alcohol, sorbitan monostearate, lactic acid, and benzyl alcohol. In yet another embodiment, the cream provided herein contains 7.7 mg ciclopirox/gram in a water miscible vanishing cream base consisting of water, octyldodecanol, mineral oil, stearyl alcohol, cocamide DEA, polysorbate 60, myristyl alcohol, sorbitan monostearate, lactic acid, and benzyl alcohol.

In yet another embodiment, the pharmaceutical compositions provided herein are formulated as a gel for topical administration, which comprise ciclopirox, or a pharmaceutically acceptable salt or solvate thereof; and one or more pharmaceutically acceptable excipients or carriers. In one embodiment, the gel provided herein comprises ciclopirox, and two or more excipients or carriers selected from the group consisting of water, isopropyl alcohol, octyldodecanol, dimethicone copolyol 190, carbomer 980, sodium hydroxide, and docusate sodium. In another embodiment, the gel provided herein comprises ciclopirox, and water, isopropyl alcohol, octyldodecanol, dimethicone copolyol 190, carbomer 980, sodium hydroxide, and docusate sodium. In yet another embodiment, the gel provided herein contains 7.7 mg ciclopirox/gram in a gel consisting of water, isopropyl alcohol, octyldodecanol, dimethicone copolyol 190, carbomer 980, sodium hydroxide, and docusate sodium.

In yet another embodiment, the pharmaceutical compositions provided herein are formulated as a shampoo for topical administration, which comprise ciclopirox, or a pharmaceutically acceptable salt or solvate thereof; and one or more pharmaceutically acceptable excipients or carriers. In one embodiment, the shampoo provided herein comprises ciclopirox, and two or more excipients or carriers selected from the group consisting of water, sodium laureth sulfate, disodium laureth sulfosuccinate, sodium chloride, and laureth-2. In another embodiment, the shampoo provided herein comprises ciclopirox, and water, sodium laureth sulfate, disodium laureth sulfosuccinate, sodium chloride, and laureth-2. In yet another embodiment, the shampoo provided herein contains 10 mg ciclopirox/gram in a shampoo base consisting of water, sodium laureth sulfate, disodium laureth sulfosuccinate, sodium chloride, and laureth-2.

In yet another embodiment, the pharmaceutical compositions provided herein are formulated as a lacquer for topical administration, which comprise ciclopirox, or a pharmaceutically acceptable salt or solvate thereof; and one or more pharmaceutically acceptable excipients or carriers. In one embodiment, the lacquer provided herein comprises ciclopirox, and two or more excipients or carriers selected from the group consisting of ethyl acetate, isopropyl alcohol, and butyl monoester of poly(methylvinyl ether/maleic acid) in isopropyl alcohol. In another embodiment, the lacquer provided herein comprises ciclopirox, and ethyl acetate, isopropyl alcohol, and butyl monoester of poly(methylvinyl ether/maleic acid) in isopropyl alcohol. In yet another embodiment, the lacquer provided herein contains 80 mg ciclopirox/gram in a solution base consisting of ethyl acetate, isopropyl alcohol, and butyl monoester of poly(methylvinyl ether/maleic acid) in isopropyl alcohol.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfate, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfate and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical compositions provided herein for inhaled/intranasal administration can further comprise a suitable flavor, such as menthol and levomenthol; and/or sweeteners, such as saccharin and saccharin sodium.

The pharmaceutical compositions provided herein for topical administration can be formulated to be immediate release or modified release, including delayed-, sustained-, pulsed-, controlled-, targeted, and programmed release.

### D. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions provided herein in a modified release dosage form can be fabricated using a matrix controlled release device known to those skilled in the art (see, Takada et al. in “Encyclopedia of Controlled Drug Delivery,” Vol. 2, Mathiowitz Ed., Wiley, 1999).

In certain embodiments, the pharmaceutical compositions provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical compositions provided herein are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical compositions provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical compositions provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874.

### Methods of Use

In one embodiment, provided herein are methods of treating leukemia in a subject, which comprises administering to the subject a therapeutically effective amount of 6-cyclohexyl-1-hydroxy-4-methyl-2(1H)-pyridone, or a pharmaceutical salt or solvate thereof.

In certain embodiments, the leukemia is relapsed leukemia. In certain embodiments, the leukemia is refractory leukemia. In certain embodiments, the leukemia is drug-resistant leukemia. In certain embodiments, the leukemia is a hereditary leukemia. In certain embodiments, the hereditary leukemia is severe congenital neutropenia (SCN). In certain embodiments, the hereditary leukemia is familial platelet disorder with acute myelogenous leukemia (FDP/AML). In certain embodiments, the leukemia is caused by LEF1. In certain embodiments, the leukemia is mediated by LEF1. In certain embodiments, the leukemia is caused by GSK3.

In certain embodiments, the leukemia is acute leukemia. In certain embodiments, the leukemia is relapsed acute leukemia. In certain embodiments, the leukemia is refractory acute leukemia. In certain embodiments, the leukemia is drug-resistant acute leukemia. In certain embodiments, the leukemia is ALL. In certain embodiments, the leukemia is relapsed ALL. In certain embodiments, the leukemia is refractory ALL. In certain embodiments, the leukemia is drug-resistant ALL. In certain embodiments, the leukemia is AML. In certain embodiments, the leukemia is relapsed AML. In certain embodiments, the leukemia is refractory AML. In certain embodiments, the leukemia is drug-resistant AML. In certain embodiments, AML has a RAS mutation. In certain embodiments, the RAS mutation is NRAS, KRAS, or HRAS. In certain embodiments, the RAS mutation is NRAS. In certain embodiments, the RAS mutation is KRAS. In certain embodiments, the RAS mutation is HRAS.

In certain embodiments, the leukemia is chronic leukemia. In certain embodiments, the leukemia is relapsed chronic leukemia. In certain embodiments, the leukemia is refractory chronic leukemia. In certain embodiments, the leukemia is drug-resistant chronic leukemia. In certain embodiments, the leukemia is CLL. In certain embodiments, the leukemia is relapsed CLL. In certain embodiments, the leukemia is refractory CLL. In certain embodiments, the leukemia is drug-resistant CLL. In certain embodiments, the leukemia is CML. In certain embodiments, the leukemia is relapsed CML. In certain embodiments, the leukemia is refractory CML. In certain embodiments, the leukemia is drug-resistant CML. In certain embodiments, the leukemia is juvenile CML. In certain embodiments, the leukemia is juvenile CML with one or more NF-1 mutations.

In certain embodiments, ALL originates in the blast cells of the bone marrow (B-cells), thymus (T-cells), or lymph nodes. ALL is categorized according to the French-American-British (FAB) Morphological Classification Scheme as L1—mature-appearing lymphoblasts (T-cells or pre-B-cells), L2—immature and pleomorphic (variously shaped) lymphoblasts (T-cells or pre-B-cells), and L3—lymphoblasts (B-cells; Burkitt's cells). In one embodiment, ALL originates in the blast cells of the bone marrow (B-cells). In another embodiment, ALL originates in the thymus (T-cells). In yet another embodiment, ALL originates in the lymph nodes. In yet another embodiment, ALL is L1 type characterized by mature-appearing lymphoblasts (T-cells or pre-B-cells). In yet another embodiment, ALL is L2 type characterized by immature and pleomorphic (variously shaped) lymphoblasts (T-cells or pre-B-cells). In still another embodiment, ALL is L3 type characterized by lymphoblasts (B-cells; Burkitt's cells).

In certain embodiments, AML is undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), or megakaryoblastic leukemia (M7). In one embodiment, AML is undifferentiated AML (M0). In another embodiment, AML is myeloblastic leukemia (M1). In yet another embodiment, AML is myeloblastic leukemia (M2). In yet another embodiment, AML is promyelocytic leukemia (M3 or M3 variant [M3V]). In yet another embodiment, AML is myelomonocytic leukemia (M4 or M4 variant with eosinophilia [M4E]). In yet another embodiment, AML is monocytic leukemia (M5). In yet another embodiment, AML is erythroleukemia (M6). In still another embodiment, AML is megakaryoblastic leukemia (M7).

In certain embodiments, the leukemia is T-cell leukemia. In one embodiment, the T-cell leukemia is peripheral T-cell leukemia, T-cell lymphoblastic leukemia, cutaneous T-cell leukemia, and adult T-cell leukemia. In another embodiment, the T-cell leukemia is peripheral T-cell leukemia. In yet another embodiment, the T-cell leukemia is T-cell lymphoblastic leukemia. In yet another embodiment, the T-cell leukemia is cutaneous T-cell leukemia. In still another embodiment, the T-cell leukemia is adult T-cell leukemia.

In certain embodiments, the leukemia is Philadelphia positive. In one embodiment, the leukemia is Philadelphia positive AML, including, but not limited to, undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), or megakaryoblastic leukemia (M7). In another embodiment, the leukemia is Philadelphia positive ALL. In yet another embodiment, the leukemia is Philadelphia positive CLL. In still another embodiment, the leukemia is Philadelphia positive CML.

In another embodiment, provided herein are methods of treating CLL in a subject, which comprises administering to the subject a therapeutically effective amount of 6-cyclohexyl-1-hydroxy-4-methyl-2(1H)-pyridone, or a pharmaceutical salt or solvate thereof.

In yet another embodiment, provided herein are methods of treating a drug-resistant hematologic malignancy in a subject, which comprises administering to the subject a therapeutically effective amount of 6-cyclohexyl-1-hydroxy-4-methyl-2(1H)-pyridone, or a pharmaceutical salt or solvate thereof.

In certain embodiments, the drug-resistant hematologic malignancy is a relapsed drug-resistant hematologic malignancy. In certain embodiments, the drug-resistant hematologic malignancy is a refractory drug-resistant hematologic malignancy. In certain embodiments, the drug-resistant hematologic malignancy is a multidrug-resistant hematologic malignancy. In certain embodiments, the drug-resistant hematologic malignancy is a Bcr-Abl kinase inhibitor-resistant hematologic malignancy. In certain embodiments, the drug-resistant hematologic malignancy is an imatinib-resistant hematologic malignancy. In certain embodiments, the drug-resistant hematologic malignancy is a dasatinib-resistant hematologic malignancy. In certain embodiments, the drug-resistant hematologic malignancy is a nilatinib-resistant hematologic malignancy. In certain embodiments, the drug-resistant hematologic malignancy is a bosutinib-resistant hematologic malignancy. In certain embodiments, the drug-resistant hematologic malignancy is a cytarabine-resistant hematologic malignancy. In certain embodiments, the drug-resistant hematologic malignancy is a vincristine-resistant hematologic malignancy.

In certain embodiments, the drug-resistant hematologic malignancy is drug-resistant myeloma, leukemia, myeloproliferative diseases, acute myeloid leukemia (AML) (including FLT3 mediated and/or KIT-mediated and/or CSF1R-mediated acute myeloid leukemia), chronic myeloid leukemias (CML) (including FLT3-mediated and/or PDGFR-mediated chronic myeloid leukemia), myelodysplastic leukemias (including FLT3-mediated myelodysplastic leukemia), myelodysplastic syndrome (including FLT3 mediated and/or Kit-mediated myelodysplastic syndrome), idiopathic hypereosinophilic syndrome (HES) (including PDGFR-mediated HES), chronic eosinophilic leukemia (CEL) (including PDGFR-mediated CEL), chronic myelomonocytic leukemia (CMML), mast cell leukemia (including Kit-mediated mast cell leukemia), or systemic mastocytosis (including Kit-mediated systemic mastocytosis).

In certain embodiments, the drug-resistant hematologic malignancy is drug-resistant lymphoma, lymphoproliferative diseases, acute lymphoblastic leukemia (ALL), B-cell acute lymphoblastic leukemias, T-cell acute lymphoblastic leukemias, chronic lymphocytic leukemia (CLL), natural killer (NK) cell leukemia, B-cell lymphoma, T-cell lymphoma, or natural killer (NK) cell lymphoma.

In certain embodiments, the drug-resistant hematologic malignancy is drug-resistant Langerhans cell histiocytosis (including CSF-1R-mediated and/or FLT3-mediated Langerhans cell histiocytosis), mast cell tumors, or mastocytosis.

In certain embodiments, the drug-resistant hematologic malignancy is drug-resistant leukemia. In certain embodiments, the drug-resistant leukemia is relapsed drug-resistant leukemia. In certain embodiments, the drug-resistant leukemia is refractory drug-resistant leukemia. In certain embodiments, the drug-resistant leukemia is multidrug-resistant leukemia. In certain embodiments, the drug-resistant leukemia is a Bcr-Abl kinase inhibitor-resistant leukemia. In certain embodiments, the drug-resistant leukemia is imatinib-resistant leukemia. In certain embodiments, the drug-resistant leukemia is dasatinib-resistant leukemia. In certain embodiments, the drug-resistant leukemia is nilatinib-resistant leukemia. In certain embodiments, the drug-resistant leukemia is bosutinib-resistant leukemia. In certain embodiments, the drug-resistant leukemia is cytarabine-resistant leukemia. In certain embodiments, the drug-resistant leukemia is vincristine-resistant leukemia.

In certain embodiments, the drug-resistant leukemia is drug-resistant acute leukemia. In certain embodiments, the drug-resistant leukemia is relapsed drug-resistant acute leukemia. In certain embodiments, the drug-resistant leukemia is refractory drug-resistant acute leukemia. In certain embodiments, the drug-resistant leukemia is multidrug-resistant acute leukemia. In certain embodiments, the drug-resistant leukemia is a Bcr-Abl kinase inhibitor-resistant acute leukemia. In certain embodiments, the drug-resistant leukemia is imatinib-resistant acute leukemia. In certain embodiments, the drug-resistant leukemia is dasatinib-resistant acute leukemia. In certain embodiments, the drug-resistant leukemia is nilatinib-resistant acute leukemia. In certain embodiments, the drug-resistant leukemia is bosutinib-resistant acute leukemia. In certain embodiments, the drug-resistant leukemia is cytarabine-resistant acute leukemia. In certain embodiments, the drug-resistant leukemia is vincristine-resistant acute leukemia.

In certain embodiments, the drug-resistant leukemia is multidrug-resistant ALL. In certain embodiments, the drug-resistant leukemia is a Bcr-Abl kinase inhibitor-resistant ALL. In certain embodiments, the drug-resistant leukemia is imatinib-resistant ALL. In certain embodiments, the drug-resistant leukemia is dasatinib-resistant ALL. In certain embodiments, the drug-resistant leukemia is nilatinib-resistant ALL. In certain embodiments, the drug-resistant leukemia is bosutinib-resistant ALL. In certain embodiments, the drug-resistant leukemia is cytarabine-resistant ALL. In certain embodiments, the drug-resistant leukemia is vincristine-resistant ALL.

In certain embodiments, the drug-resistant leukemia is multidrug-resistant AML. In certain embodiments, the drug-resistant leukemia is a Bcr-Abl kinase inhibitor-resistant AML. In certain embodiments, the drug-resistant leukemia is imatinib-resistant AML. In certain embodiments, the drug-resistant leukemia is dasatinib-resistant AML. In certain embodiments, the drug-resistant leukemia is nilatinib-resistant AML. In certain embodiments, the drug-resistant leukemia is bosutinib-resistant AML. In certain embodiments, the drug-resistant leukemia is cytarabine-resistant AML. In certain embodiments, the drug-resistant leukemia is vincristine-resistant AML.

In certain embodiments, the drug-resistant leukemia is drug-resistant chronic leukemia. In certain embodiments, the drug-resistant leukemia is relapsed drug-resistant chronic leukemia. In certain embodiments, the drug-resistant leukemia is refractory drug-resistant chronic leukemia. In certain embodiments, the drug-resistant leukemia is multidrug-resistant chronic leukemia. In certain embodiments, the drug-resistant leukemia is a Bcr-Abl kinase inhibitor-resistant chronic leukemia. In certain embodiments, the drug-resistant leukemia is imatinib-resistant chronic leukemia. In certain embodiments, the drug-resistant leukemia is dasatinib-resistant chronic leukemia. In certain embodiments, the drug-resistant leukemia is nilatinib-resistant chronic leukemia. In certain embodiments, the drug-resistant leukemia is bosutinib-resistant chronic leukemia. In certain embodiments, the drug-resistant leukemia is cytarabine-resistant chronic leukemia. In certain embodiments, the drug-resistant leukemia is vincristine-resistant chronic leukemia.

In certain embodiments, the drug-resistant leukemia is multidrug-resistant CLL. In certain embodiments, the drug-resistant leukemia is a Bcr-Abl kinase inhibitor-resistant CLL. In certain embodiments, the drug-resistant leukemia is imatinib-resistant CLL. In certain embodiments, the drug-resistant leukemia is dasatinib-resistant CLL. In certain embodiments, the drug-resistant leukemia is nilatinib-resistant CLL. In certain embodiments, the drug-resistant leukemia is bosutinib-resistant CLL. In certain embodiments, the drug-resistant leukemia is cytarabine-resistant CLL. In certain embodiments, the drug-resistant leukemia is vincristine-resistant CLL.

In certain embodiments, the drug-resistant leukemia is multidrug-resistant CML. In certain embodiments, the drug-resistant leukemia is a Bcr-Abl kinase inhibitor-resistant CML. In certain embodiments, the drug-resistant leukemia is imatinib-resistant CML. In certain embodiments, the drug-resistant leukemia is dasatinib-resistant CML. In certain embodiments, the drug-resistant leukemia is nilatinib-resistant CML. In certain embodiments, the drug-resistant leukemia is bosutinib-resistant CML. In certain embodiments, the drug-resistant leukemia is cytarabine-resistant CML. In certain embodiments, the drug-resistant leukemia is vincristine-resistant CML.

In certain embodiments, the drug-resistant leukemia is Philadelphia positive. In one embodiment, the drug-resistant leukemia is Philadelphia positive AML, including, but not limited to, undifferentiated AML (M0), myeloblastic leukemia (M1), myeloblastic leukemia (M2), promyelocytic leukemia (M3 or M3 variant [M3V]), myelomonocytic leukemia (M4 or M4 variant with eosinophilia [M4E]), monocytic leukemia (M5), erythroleukemia (M6), or megakaryoblastic leukemia (M7). In another embodiment, the drug-resistant leukemia is Philadelphia positive ALL. In yet another embodiment, the drug-resistant leukemia is Philadelphia positive CLL. In still another embodiment, the drug-resistant leukemia is Philadelphia positive CML.

In yet another embodiment, provided herein are methods of treating drug-resistant leukemia in a subject, which comprises administering to the subject a therapeutically effective amount of 6-cyclohexyl-1-hydroxy-4-methyl-2(1H)-pyridone, or a pharmaceutical salt or solvate thereof. In one embodiment, the leukemia is resistant to a Bcr-Abl kinase inhibitor. In another embodiment, the leukemia is resistant to imatinib, dasatinib, nilatinib, bosutinib, cytarabine, or vincristine. In yet another embodiment, the leukemia is resistant to dasatinib, nilatinib, bosutinib, cytarabine, or vincristine.

In yet another embodiment, provided herein are methods of treating drug-resistant acute leukemia in a subject, which comprises administering to the subject a therapeutically effective amount of 6-cyclohexyl-1-hydroxy-4-methyl-2(1H)-pyridone, or a pharmaceutical salt or solvate thereof. In one embodiment, the acute leukemia is resistant to a Bcr-Abl kinase inhibitor. In another embodiment, the acute leukemia is resistant to imatinib, dasatinib, nilatinib, bosutinib, cytarabine, or vincristine. In yet another embodiment, the acute leukemia is resistant to dasatinib, nilatinib, bosutinib, cytarabine, or vincristine.

In yet another embodiment, provided herein are methods of treating drug-resistant ALL in a subject, which comprises administering to the subject a therapeutically effective amount of 6-cyclohexyl-1-hydroxy-4-methyl-2(1H)-pyridone, or a pharmaceutical salt or solvate thereof. In one embodiment, ALL is resistant to a Bcr-Abl kinase inhibitor. In another embodiment, ALL is resistant to imatinib, dasatinib, nilatinib, bosutinib, cytarabine, or vincristine. In yet another embodiment, ALL is resistant to dasatinib, nilatinib, bosutinib, cytarabine, or vincristine.

In yet another embodiment, provided herein are methods of treating drug-resistant AML in a subject, which comprises administering to the subject a therapeutically effective amount of 6-cyclohexyl-1-hydroxy-4-methyl-2(1H)-pyridone, or a pharmaceutical salt or solvate thereof. In one embodiment, AML is resistant to a Bcr-Abl kinase inhibitor.

In another embodiment, AML is resistant to imatinib, dasatinib, nilatinib, bosutinib, cytarabine, or vincristine. In yet another embodiment, AML is resistant to dasatinib, nilatinib, bosutinib, cytarabine, or vincristine.

In yet another embodiment, provided herein are methods of treating drug-resistant chronic leukemia in a subject, which comprises administering to the subject a therapeutically effective amount of 6-cyclohexyl-1-hydroxy-4-methyl-2(1H)-pyridone, or a pharmaceutical salt or solvate thereof. In one embodiment, the chronic leukemia is resistant to a Bcr-Abl kinase inhibitor. In another embodiment, the chronic leukemia is resistant to imatinib, dasatinib, nilatinib, bosutinib, cytarabine, or vincristine. In yet another embodiment, the chronic leukemia is resistant to dasatinib, nilatinib, bosutinib, cytarabine, vincristine.

In yet another embodiment, provided herein are methods of treating drug-resistant CLL in a subject, which comprises administering to the subject a therapeutically effective amount of 6-cyclohexyl-1-hydroxy-4-methyl-2(1H)-pyridone, or a pharmaceutical salt or solvate thereof. In one embodiment, CLL is resistant to a Bcr-Abl kinase inhibitor. In another embodiment, CLL is resistant to imatinib, dasatinib, nilatinib, bosutinib, cytarabine, or vincristine. In yet another embodiment, CLL is resistant to dasatinib, nilatinib, bosutinib, cytarabine, or vincristine.

In yet another embodiment, provided herein are methods of treating drug-resistant CML in a subject, which comprises administering to the subject a therapeutically effective amount of 6-cyclohexyl-1-hydroxy-4-methyl-2(1H)-pyridone, or a pharmaceutical salt or solvate thereof. In one embodiment, CML is resistant to a Bcr-Abl kinase inhibitor.

In another embodiment, CML is resistant to imatinib, dasatinib, nilatinib, bosutinib, cytarabine, or vincristine. In yet another embodiment, CML is resistant to dasatinib, nilatinib, bosutinib, cytarabine, or vincristine.

In certain embodiments, the therapeutically effective amount is ranging from about 0.1 to about 100 mg/kg/day, from about 0.1 to about 50 mg/kg/day, from about 0.1 to about 40 mg/kg/day, from about 0.1 to about 30 mg/kg/day, from about 0.1 to about 25 mg/kg/day, from about 0.1 to about 20 mg/kg/day, from about 0.1 to about 15 mg/kg/day, from about 0.1 to about 10 mg/kg/day, or from about 0.1 to about 5 mg/kg/day. In one embodiment, the therapeutically effective amount is ranging from about 0.1 to about 100 mg/kg/day. In another embodiment, the therapeutically effective amount is ranging from about 0.1 to about 50 mg/kg/day. In yet another embodiment, the therapeutically effective amount is ranging from about 0.1 to about 40 mg/kg/day. In yet another embodiment, the therapeutically effective amount is ranging from about 0.1 to about 30 mg/kg/day. In yet another embodiment, the therapeutically effective amount is ranging from about 0.1 to about 25 mg/kg/day. In yet another embodiment, the therapeutically effective amount is ranging from about 0.1 to about 20 mg/kg/day. In yet another embodiment, the therapeutically effective amount is ranging from about 0.1 to about 15 mg/kg/day. In yet another embodiment, the therapeutically effective amount is ranging from about 0.1 to about 10 mg/kg/day. In still another embodiment, the therapeutically effective amount is ranging from about 0.1 to about 5 mg/kg/day.

In certain embodiments, ciclopirox, or a pharmaceutically acceptable salt or solvate thereof, is administered orally. In certain embodiments, the therapeutically effective amount for oral administration ranges from about 0.1 to about 100, from about 0.5 to about 50, or from about 1 to about 25 mg/kg/day. In certain embodiments, the therapeutically effective amount for oral administration is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 mg/kg/day. In certain embodiments, the therapeutically effective amount for oral administration is about 2, about 5, about 10, about 15, or about 20 mg/kg/day. In certain embodiments, the therapeutically effective amount for oral administration is about 1 mg/kg/day. In certain embodiments, the therapeutically effective amount for oral administration is about 2 mg/kg/day. In certain embodiments, the therapeutically effective amount for oral administration is about 5 mg/kg/day. In certain embodiments, the therapeutically effective amount for oral administration is about 10 mg/kg/day. In certain embodiments, the therapeutically effective amount for oral administration is about 15 mg/kg/day. In certain embodiments, the therapeutically effective amount for oral administration is about 20 mg/kg/day.

In certain embodiments, ciclopirox, or a pharmaceutically acceptable salt or solvate thereof, is administered intravenously. In certain embodiments, the therapeutically effective amount for oral administration ranges from about 0.001 to about 20, from about 0.01 to about 10, from about 0.01 to about 5, from about 0.05 to about 1 mg/kg/day, about 0.05 to about 0.95, or from about 0.05 to about 0.90 mg/kg/day. In certain embodiments, the therapeutically effective amount for intravenous administration is about 0.05, about 0.06, about 0.08, about 0.1, about 0.15, about 0.2, about 0.3, about 0.4, about 0.5, about 0.6, about 0.7, about 0.8, about 0.9, about 0.95, about 0.99, or about 1 mg/kg/day. In certain embodiments, the therapeutically effective amount for intravenous administration is about 0.1 mg/kg/day. In certain embodiments, the therapeutically effective amount for intravenous administration is about 0.2 mg/kg/day. In certain embodiments, the therapeutically effective amount for intravenous administration is about 0.3 mg/kg/day. In certain embodiments, the therapeutically effective amount for intravenous administration is about 0.5 mg/kg/day.

It is understood that the administered dose can also be expressed in units other than mg/kg/day. For example, doses for parenteral administration can be expressed as mg/m²/day. One of ordinary skill in the art would readily know how to convert doses from mg/kg/day to mg/m²/day to given either the height or weight of a subject or both (see, www.fda.gov/cder/cancer/animalframe.htm). For example, a dose of 1 mg/m²/day for a 65 kg human is approximately equal to 38 mg/kg/day.

In yet another embodiment, provided herein is a method of treating a leukemia or drug-resistant hematologic malignancy in a subject, which comprises administering to the subject ciclopirox, or a therapeutically acceptable salt or solvate thereof, in an amount sufficient to provide a plasma concentration of ciclopirox at steady state, ranging from about 0.01 to about 100 μM, from about 0.1 to about 50 μM, from about 0.2 to about 20 μM, from about 1 to about 20 μM, or from about 5 to about 20 μM. In one embodiment, the amount of ciclopirox administered is sufficient to provide a plasma concentration of ciclopirox at steady state ranging from about 1 to about 20 μM. In another embodiment, the amount of ciclopirox administered is sufficient to provide a plasma concentration of ciclopirox at steady state of about 1, about 2, about 5, about 10, or about 20 μM. As used herein, the term “plasma concentration at steady state” is the concentration reached after a period of administration of a compound. Once steady state is reached, there are minor peaks and troughs on the time dependent curve of the plasma concentration of the compound.

In yet another embodiment, provided herein is a method of treating a leukemia or drug-resistant hematologic malignancy in a subject, which comprises administering to the subject ciclopirox, or a therapeutically acceptable salt or solvate thereof, in an amount sufficient to provide a maximum plasma concentration (peak concentration) of ciclopirox ranging from about 0.01 to about 100 μM, from about 0.1 to about 50 μM, from about 0.2 to about 20 μM, from about 1 to about 20 μM, or from about 5 to about 20 μM. In one embodiment, the amount of ciclopirox administered is sufficient to provide a maximum plasma concentration ranging from about 1 to about 50 μM. In another embodiment, the amount of ciclopirox administered is sufficient to provide a maximum plasma concentration of about 1, about 2, about 5, about 10, about 15, about 20, about 25, about 30, about 40, or about 50 μM.

In yet another embodiment, provided herein is a method of treating a leukemia or drug-resistant hematologic malignancy in a subject, which comprises administering to the subject ciclopirox, or a therapeutically acceptable salt or solvate thereof, in an amount sufficient to provide a maximum plasma concentration (peak concentration) of ciclopirox ranging from about 0.01 to about 100 μM, from about 0.1 to about 50 μM, from about 0.2 to about 20 μM, from about 1 to about 20 μM, or from about 5 to about 20 μM, when two or more doses of 6-cyclohexyl-1-hydroxy-4-methyl-2(1H)-pyridone are administered. In one embodiment, the amount of ciclopirox administered is sufficient to provide a maximum plasma concentration ranging from about 1 to about 50 μM, when two or more doses of 6-cyclohexyl-1-hydroxy-4-methyl-2(1H)-pyridone are administered. In another embodiment, the amount of ciclopirox administered is sufficient to provide a maximum plasma concentration of about 1, about 2, about 5, about 10, about 15, about 20, about 25, about 30, about 40, or about 50 μM, when two or more doses of 6-cyclohexyl-1-hydroxy-4-methyl-2(1H)-pyridone are administered.

In yet another embodiment, provided herein is a method of treating a leukemia or drug-resistant hematologic malignancy in a subject, which comprises administering to the subject ciclopirox, or a therapeutically acceptable salt or solvate thereof, wherein ciclopirox has a half life (t_{1/2}) ranging from about 1 to about 200 hrs, from about 2 to about 100 hrs, from about 5 to about 50 hrs, from about 5 to 25 hrs, or from about 5 to 10 hrs. In one embodiment, ciclopirox has a half life (t_{1/2}) of about 5, about 7, about 10, about 15, about 20, about 40, about 60, about 80, or about 100 hrs.

In certain embodiments, the subject is a mammal. In certain embodiments, the subject is a human.

In certain embodiments, the subject to be treated with one of the methods provided herein has not been treated with anticancer therapy for the leukemia or drug-resistant hematologic malignancy to be treated prior to the administration of ciclopirox, or a pharmaceutically acceptable salt or solvate thereof.

In certain embodiments, the subject to be treated with one of the methods provided herein has been treated with anticancer therapy for the leukemia or drug-resistant hematologic malignancy to be treated prior to the administration of ciclopirox, or a pharmaceutically acceptable salt or solvate thereof.

In certain embodiments, the subject to be treated with one of the methods provided herein is an age ranging from about 1 to about 100 yrs, from about 1 to about 10 yrs, from about 1 to about 15 yrs, from about 1 to about 20 yrs, from about 10 to about 20 yrs, from about 15 to about 85 yrs, from about 40 to about 85 yrs, or from about 55 to about 85 yrs.

The methods provided herein encompass treating a subject regardless of patient's age, although some diseases or disorders are more common in certain age groups. Further provided herein is a method for treating a subject who has undergone surgery in an attempt to treat the disease or condition at issue, as well as the one who have not. Because the subjects with cancer have heterogeneous clinical manifestations and varying clinical outcomes, the treatment given to a particular subject may vary, depending on his/her prognosis. The skilled clinician will be able to readily determine without undue experimentation, specific secondary agents, types of surgery, and types of non-drug based standard therapy that can be effectively used to treat an individual subject with cancer.

Depending on the disease to be treated and the subject's condition, ciclopirox, or a pharmaceutically acceptable salt or solvate, may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) route of administration.

Ciclopirox, or a pharmaceutically acceptable salt or solvate thereof, may be formulated, alone or together, in suitable dosage unit with pharmaceutically acceptable excipients, carriers, adjuvants and vehicles, appropriate for each route of administration.

In one embodiment, ciclopirox, or a pharmaceutically acceptable salt or solvate thereof, is administered orally. In another embodiment, ciclopirox, or a pharmaceutically acceptable salt or solvate thereof, is administered parenterally. In yet another embodiment, ciclopirox, or a pharmaceutically acceptable salt or solvate thereof, is administered intravenously. In yet another embodiment, ciclopirox, or a pharmaceutically acceptable salt or solvate thereof, is administered intramuscularly. In yet another embodiment, ciclopirox, or a pharmaceutically acceptable salt or solvate thereof, is administered subcutaneously. In still another embodiment, ciclopirox, or a pharmaceutically acceptable salt or solvate thereof, is administered topically.

In certain embodiments, ciclopirox, or a pharmaceutically acceptable salt or solvate thereof, is delivered as a single dose, in one embodiment, a single bolus injection, in another embodiment, oral tablets or pills. In certain embodiments, ciclopirox, or a pharmaceutically acceptable salt or solvate thereof, is delivered or over time, in one embodiment, continuous infusion over time, in another embodiment, divided bolus doses over time.

Ciclopirox, or a pharmaceutically acceptable salt or solvate thereof, can be administered repetitively if necessary, for example, until the patient experiences stable disease or regression, or until the patient experiences disease progression or unacceptable toxicity. For example, stable disease for solid tumors generally means that the perpendicular diameter of measurable lesions has not increased by 25% or more from the last measurement. Response Evaluation Criteria in Solid Tumors (RECIST) Guidelines, Journal of the National Cancer Institute 92(3): 205-216 (2000). Stable disease or lack thereof is determined by methods known in the art such as evaluation of patient symptoms, physical examination, visualization of the tumor that has been imaged using X-ray, CAT, PET, or MRI scan and other commonly accepted evaluation modalities.

Ciclopirox, or a pharmaceutically acceptable salt or solvate thereof, can be administered once daily (QD), or divided into multiple daily doses such as twice daily (BID), and three times daily (TID). In addition, the administration can be continuous, i.e., every day, or intermittently. The term “intermittent” or “intermittently” as used herein is intended to mean stopping and starting at either regular or irregular intervals. For example, intermittent administration of ciclopirox, or a pharmaceutically acceptable salt or solvate thereof, is administration for one to six days per week, administration in cycles (e.g., daily administration for two to eight consecutive weeks, then a rest period with no administration for up to one week), or administration on alternate days. In certain embodiments, ciclopirox, or 6-cyclohexyl-1-hydroxy-4-methyl-2(1H)-pyridone, or a pharmaceutical salt or solvate thereof, or a pharmaceutically acceptable salt or solvate thereof, is administered once per day, twice per day, or three times per day for about 1 to about 26 six weeks.

In certain embodiments, ciclopirox, or a pharmaceutically acceptable salt or solvate thereof, is cyclically administered to a patient. Cycling therapy involves the administration of ciclopirox, or a pharmaceutically acceptable salt or solvate thereof, for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

In certain embodiments, the frequency of administration of ciclopirox, or a pharmaceutically acceptable salt or solvate thereof, is in the range of about a daily dose to about a monthly dose. In certain embodiments, administration is once a day, twice a day, three times a day, four times a day, once every other day, twice a week, once every week, once every two weeks, once every three weeks, or once every four weeks. In one embodiment, ciclopirox, or a pharmaceutically acceptable salt or solvate thereof, is administered once a day. In another embodiment, ciclopirox, or a pharmaceutically acceptable salt or solvate thereof, is administered twice a day. In yet another embodiment, ciclopirox, or a pharmaceutically acceptable salt or solvate thereof, is administered three times a day. In still another embodiment, ciclopirox, or a pharmaceutically acceptable salt or solvate thereof, is administered four times a day.

In certain embodiments, ciclopirox, or a pharmaceutically acceptable salt or solvate thereof, is administered once per day from one day to six months, from one week to three months, from one week to four weeks, from one week to three weeks, or from one week to two weeks. In certain embodiments, ciclopirox, or a pharmaceutically acceptable salt or solvate thereof, is administered once per day for about 1 week, 2 weeks, 3 weeks, about 4 weeks, about 6 weeks, about 9 weeks, about 12 weeks, about 15 weeks, about 18 weeks, about 21 weeks, or about 26 weeks.

In certain embodiments, ciclopirox, or a pharmaceutically acceptable salt or solvate thereof, is administered intermittently. In certain embodiments, ciclopirox, or a pharmaceutically acceptable salt or solvate thereof, is administered continuously. In certain embodiments, ciclopirox, or a pharmaceutically acceptable salt or solvate thereof, is cyclically administered to a patient.

In certain embodiments, ciclopirox, or a pharmaceutically acceptable salt or solvate thereof, is administered daily in a single or divided doses for one week, two weeks, three weeks, four weeks, five weeks, six weeks, eight weeks, ten weeks, fifteen weeks, or twenty weeks, followed by a rest period of about 1 day to about ten weeks. For example, the methods contemplate using cycling of one week, two weeks, three weeks, four weeks, five weeks, six weeks, eight weeks, ten weeks, fifteen weeks, or twenty weeks. In certain embodiments, ciclopirox, or a pharmaceutically acceptable salt or solvate thereof, is administered daily in a single or divided doses for one week, two weeks, three weeks, four weeks, five weeks, or six weeks with a rest period of 1, 3, 5, 7, 9, 12, 14, 16, 18, 20, 22, 24, 26, 28, 29, or 30 days. In certain embodiments, the rest period is 14 days. In certain embodiments, the rest period is 28 days. In one embodiment, the rest period is a period that is sufficient for bone marrow recovery. The frequency, number and length of dosing cycles can be increased or decreased.

In certain embodiments, the methods provided herein comprise: i) administering to the subject at a first daily dose of ciclopirox, or a pharmaceutically acceptable salt or solvate thereof, ii) resting for a period of at least one day where ciclopirox, or a pharmaceutically acceptable salt or solvate thereof, is not administered to the subject; iii) administering a second dose of ciclopirox, or a pharmaceutically acceptable salt or solvate thereof, to the subject; and iv) repeating steps ii) to iii) a plurality of times.

Ciclopirox, or a pharmaceutically acceptable salt or solvate thereof, can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein also are kits which, when used by the medical practitioner, can simplify the administration of appropriate amounts of active ingredients to a subject. In certain embodiments, the kit provided herein includes a container and a dosage form of ciclopirox, or a pharmaceutically acceptable salt or solvate thereof. In certain embodiments, the kit includes a container comprising a dosage form of ciclopirox, or a pharmaceutically acceptable salt or solvate thereof.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

In certain embodiments, provided herein is a method of inhibiting the growth of a leukemia stem cell, comprising the step of contacting the leukemia stem cell with ciclopirox, or a pharmaceutically acceptable salt or solvate thereof. In certain embodiment, the effective amount of ciclopirox ranges from about 1 μM to about 1 mM, from about 10 μM to about 10 μM, from about 100 μM to about 2 μM, or from about 1 nM to about 1 μM.

In certain embodiments, the leukemia stem cell is a relapsed leukemia step cell. In certain embodiments, the leukemia stem cell is a refractory leukemia step cell. In certain embodiments, the leukemia stem cell is a drug-resistant leukemia stem cell. In certain embodiments, the leukemia stem cell is a multidrug-resistant leukemia stem cell. In certain embodiments, the leukemia stem cell is a Bcr-Abl kinase inhibitor-resistant leukemia stem cell. In certain embodiments, the leukemia stem cell is an imatinib-resistant leukemia stem cell. In certain embodiments, the leukemia stem cell is a dasatinib-resistant leukemia stem cell. In certain embodiments, the leukemia stem cell is a nilatinib-resistant leukemia stem cell. In certain embodiments, the leukemia stem cell is a bosutinib-resistant leukemia stem cell. In certain embodiments, the leukemia stem cell is a cytarabine-resistant leukemia stem cell. In certain embodiments, the leukemia stem cell is a vincristine-resistant leukemia stem cell.

In certain embodiment, the leukemia stem cell is an acute leukemia stem cell. In certain embodiments, the leukemia stem cell is a relapsed acute leukemia stem cell. In certain embodiments, the leukemia stem cell is a refractory acute leukemia stem cell. In certain embodiments, the leukemia stem cell is a drug-resistant acute leukemia stem cell. In certain embodiments, the leukemia stem cell is a multidrug-resistant acute leukemia stem cell. In certain embodiments, the leukemia stem cell is a Bcr-Abl kinase inhibitor-resistant acute leukemia stem cell. In certain embodiments, the leukemia stem cell is an imatinib-resistant acute leukemia stem cell. In certain embodiments, the leukemia stem cell is a dasatinib-resistant acute leukemia stem cell. In certain embodiments, the leukemia stem cell is a nilatinib-resistant acute leukemia stem cell. In certain embodiments, the leukemia stem cell is a bosutinib-resistant acute leukemia stem cell. In certain embodiments, the leukemia stem cell is a cytarabine-resistant acute leukemia stem cell. In certain embodiments, the leukemia stem cell is a vincristine-resistant acute leukemia stem cell.

In certain embodiments, the leukemia stem cell is an ALL stem cell. In certain embodiments, the leukemia stem cell is a relapsed ALL stem cell. In certain embodiments, the leukemia stem cell is a refractory ALL stem cell. In certain embodiments, the leukemia stem cell is a drug-resistant ALL stem cell. In certain embodiments, the leukemia stem cell is a multidrug-resistant ALL stem cell. In certain embodiments, the leukemia stem cell is a Bcr-Abl kinase inhibitor-resistant ALL stem cell. In certain embodiments, the leukemia stem cell is an imatinib-resistant ALL stem cell. In certain embodiments, the leukemia stem cell is a dasatinib-resistant ALL stem cell. In certain embodiments, the leukemia stem cell is a nilatinib-resistant ALL stem cell. In certain embodiments, the leukemia stem cell is a bosutinib-resistant ALL stem cell. In certain embodiments, the leukemia stem cell is a cytarabine-resistant ALL stem cell. In certain embodiments, the leukemia stem cell is a vincristine-resistant ALL stem cell.

In certain embodiments, the leukemia stem cell is an AML stem cell. In certain embodiments, the leukemia stem cell is a relapsed AML stem cell. In certain embodiments, the leukemia stem cell is a refractory AML stem cell. In certain embodiments, the leukemia stem cell is a drug-resistant AML stem cell. In certain embodiments, the leukemia stem cell is a multidrug-resistant AML stem cell. In certain embodiments, the leukemia stem cell is a Bcr-Abl kinase inhibitor-resistant AML stem cell. In certain embodiments, the leukemia stem cell is an imatinib-resistant AML stem cell. In certain embodiments, the leukemia stem cell is a dasatinib-resistant AML stem cell. In certain embodiments, the leukemia stem cell is a nilatinib-resistant AML stem cell. In certain embodiments, the leukemia stem cell is a bosutinib-resistant AML stem cell. In certain embodiments, the leukemia stem cell is a cytarabine-resistant AML stem cell. In certain embodiments, the leukemia stem cell is a vincristine-resistant AML stem cell.

In certain embodiment, the leukemia stem cell is a chronic leukemia cell. In certain embodiments, the leukemia stem cell is a relapsed chronic leukemia cell. In certain embodiments, the leukemia stem cell is a refractory chronic leukemia cell. In certain embodiments, the leukemia stem cell is a drug-resistant chronic leukemia cell. In certain embodiments, the leukemia stem cell is a multidrug-resistant chronic leukemia cell. In certain embodiments, the leukemia stem cell is a Bcr-Abl kinase inhibitor-resistant chronic leukemia cell. In certain embodiments, the leukemia stem cell is an imatinib-resistant chronic leukemia cell. In certain embodiments, the leukemia stem cell is a dasatinib-resistant chronic leukemia cell. In certain embodiments, the leukemia stem cell is a nilatinib-resistant chronic leukemia cell. In certain embodiments, the leukemia stem cell is a bosutinib-resistant chronic leukemia cell. In certain embodiments, the leukemia stem cell is a cytarabine-resistant chronic leukemia cell. In certain embodiments, the leukemia stem cell is a vincristine-resistant chronic leukemia cell.

In certain embodiments, the leukemia stem cell is a CLL stem cell. In certain embodiments, the leukemia stem cell is a relapsed CLL stem cell. In certain embodiments, the leukemia stem cell is a refractory CLL stem cell. In certain embodiments, the leukemia stem cell is a drug-resistant CLL stem cell. In certain embodiments, the leukemia stem cell is a multidrug-resistant CLL stem cell. In certain embodiments, the leukemia stem cell is a Bcr-Abl kinase inhibitor-resistant CLL stem cell. In certain embodiments, the leukemia stem cell is an imatinib-resistant CLL stem cell. In certain embodiments, the leukemia stem cell is a dasatinib-resistant CLL stem cell. In certain embodiments, the leukemia stem cell is a nilatinib-resistant CLL stem cell. In certain embodiments, the leukemia stem cell is a bosutinib-resistant CLL stem cell. In certain embodiments, the leukemia stem cell is a cytarabine-resistant CLL stem cell. In certain embodiments, the leukemia stem cell is a vincristine-resistant CLL stem cell.

In certain embodiments, the leukemia stem cell is a CML stem cell. In certain embodiments, the leukemia stem cell is a relapsed CML stem cell. In certain embodiments, the leukemia stem cell is a refractory CML stem cell. In certain embodiments, the leukemia stem cell is a drug-resistant CML stem cell. In certain embodiments, the leukemia stem cell is a multidrug-resistant CML stem cell. In certain embodiments, the leukemia stem cell is a Bcr-Abl kinase inhibitor-resistant CML stem cell. In certain embodiments, the leukemia stem cell is an imatinib-resistant CML stem cell. In certain embodiments, the leukemia stem cell is a dasatinib-resistant CML stem cell. In certain embodiments, the leukemia stem cell is a nilatinib-resistant CML stem cell. In certain embodiments, the leukemia stem cell is a bosutinib-resistant CML stem cell. In certain embodiments, the leukemia stem cell is a cytarabine-resistant CML stem cell. In certain embodiments, the leukemia stem cell is a vincristine-resistant CML stem cell.

In certain embodiments, the leukemia stem cell is Philadelphia positive leukemia stem cell. In one embodiment, the leukemia stem cell is a Philadelphia positive ALL stem cell. In another embodiment, the leukemia stem cell is a Philadelphia positive AML stem cell. In yet another embodiment, the leukemia stem cell is a Philadelphia positive CLL stem cell. In still another embodiment, the leukemia stem cell is a Philadelphia positive CML stem cell.

The inhibition of cell growth can be gauged by, e.g., counting the number of cells contacted with a compound of interest, comparing the cell proliferation with otherwise identical cells not contacted with the compound, or determining the size of the tumor that encompasses the cells. The number of cells, as well as the size of the cells, can be readily assessed using any method known in the art (e.g., trypan blue exclusion and cell counting, measuring incorporation of ³H-thymidine into nascent DNA in a cell).

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); mM (millimolar); μM (micromolar); Hz (Hertz); MHz (megahertz); mmol (millimoles); hr or hrs (hours); min (minutes); and DMSO (dimethylsulfoxide).

For all of the following examples, standard procedures known to those skilled in the art can be utilized. Unless otherwise indicated, all temperatures are expressed in ° C. (degrees Centigrade). All procedures are conducted at room temperature unless otherwise noted. Methodologies illustrated herein are intended to exemplify the applicable science through the use of specific examples and are not indicative of the scope of the disclosure.

### Example 1

### General Biological Methods

### Cell Culture

Leukemia cells or cell lines (HL-60, RSV411, k562, Jurkat, U937), lymphoma cells or cellines (MDAY-D2), solid tumor cells or cell lines (PPC-1, HeLa, OVCAR-3, DU-145, HT-29), and GMO5757 human lung fibroblasts, were cultured in RPMI 1640 medium. HepG2 hepatoma cells and MRC5 human lung fibroblasts were grown in Dulbecco modified Eagle medium. OCI-M2, OCI-AML2, and NB4 leukemia cell lines and OPM2, KMS11, LP1, UTMC2, KSM18, and OCIMy5 myeloma cell lines were maintained in Iscove Modified Dulbecco Medium. LF1 human lung fibroblasts were maintained in HAM medium. All media were supplemented with 10% fetal calf serum, 100 μg/mL of penicillin, and 100 units/mL of streptomycin (all from Hyclone, Logan, Utah). The cells were incubated at 37° C. in a humidified air atmosphere supplemented with 5% CO₂.

### Cell Cycle

Cells were harvested, washed with cold PBS, resuspended in 70% cold ethanol, and incubated overnight at −20° C. Cells were then treated with 100 ng/mL of DNase-free RNase (Invitrogen, Carlsbad, Calif.) at 37° C. for 30 min, washed with cold PBS, and resuspended in PBS with 50 μg/mL of protease inhibitors (Sigma). DNA content was analyzed by flow cytometry (FACSCalibur; BD Biosciences, San Jose, Calif.).

### Example 2

### Luciferase Assay for Anti-Cancer Activity

The anticancer activity of ciclopirox was determined using the luciferase assay as described herein.

For the luciferase assay, HeLa cells that stably over-express the human survivin promoter driving firefly luciferase were used, which were prepared by first isolating the full-length survivin promoter (−1059 upstream of the initiating ATG) from HeLa genomic DNA using the forward primer 5′-GGCGAGCTCACTTTTTCTGTCACCTCCGTGGTCCG-3′ (SEQ ID NO: 1) and the reverse primer 5′-GGGTTCGAAACGGCGGCGGCGGTGGAGA-3′ (SEQ ID NO:2). The survivin promoter was then sub-cloned into the GL4.20 firefly luciferase reporter vector (Promega Corporation, Madison, Wis.). Clones were sequence-verified for orientation and integrity using a CEQ 8000 Genetic Analysis System (Beckman, Mississauga, ON, Canada). HeLa cells were transfected with survivin promoter construct alone or vector alone using Lipofectamine (Invitrogen, CA), and selected with Puromycin (4 μg/mL) (Sigma) for stable clones. Stable HeLa cells thus selected were used for testing ciclopirox for its anticancer activity.

To determine anticancer activity, HeLa cells stably over-expressing the human survivin promoter driving firefly luciferase were treated with ciclopirox at 5 μM for 24 hrs. The HeLa cells (15,000 cells/well) were plated in 96-well plates. After adhering to the plates, the HeLa cells were treated with ciclopirox at 5 μM (0.05% DMSO). After 24-hr incubation, survivin promoter activity was assessed using a luciferase assay to assess the inhibition of transactivation of the survivin promoter. During the measurement, cell culture medium was removed from a 96-well plate and 1× GIo Lysis buffer (Promega) was added to the plate. After 10-min incubation, an equal volume of Bright-Glo Luciferase substrate (Promega) was added, and the luminescence signal was detected with a 96-well Luminoskan luminescence plate reader (Thermo Fisher Scientific, Waltham, Mass.) with 5-seconds integration time.

Ciclopirox was retested for reproducibility using the luciferase assay and was also tested for viability. Cell viability was determined using the CellTiter96 aqueous nonradioactive (MTS) assay, where propidium iodide (PI) staining was used (Biovision, Mountain view, Calif.).

Ciclopirox was further evaluated as an anti-cancer agent by treating leukemia cell lines with increasing concentrations of the compound for 72 hrs. Cell viability was also measured by the MTS assay. Cell death was evaluated by detecting the presence of a subG1 peak by flow cytometry after staining cells with PI.

Results were normalized and corrected for systematic errors using the B score (Gunter, J. Biomol. Screen. 2003, 8, 624-633).

Ciclopirox repressed survivin transactivation greater than 60% while maintaining greater than 90% cell viability at 24 hours after treatment. The effect of ciclopirox on survivin transactivation was confirmed in a dose response study.

### Example 3

### Determination of Survivin mRNA and Protein Expression Levels in HeLa Cells

The survivin mRNA and protein expression levels in wild type HeLa cells that were treated with ciclopirox were determined using quantitative real-time polymerase chain reaction (QRT-PCR) and immunoblotting to determine its anticancer activity.

For QRT-PCR, cDNAs encoding survivin and glyceraldehyde-3-phosphate dehydrogenase (GAPDH) were amplified using the following primer pairs: survivin, forward, 5′-TTTTCATCGTCGTCCCTAGC-3′ (SEQ ID NO:3); reverse, 5′-CGACTCAGATGTGGCAGAAA-3′ (SEQ ID NO:4); and GAPDH, forward, 5′-GAAGGTGAAGGTCGGAGTC-3″ (SEQ ID NO:5); reverse, 5′-GAAGATGGTGATGGGATTTC-3′ (SEQ ID NO:6). Equal amounts of cDNAs were added to a prepared master mix (SYBR Green PCR Master mix; Applied Biosystems, Foster City, Calif.). QRT-PCR is performed on an ABI Prism 7700 sequence detection system (Applied Biosystems, Foster City, Calif.). The relative abundance of a transcript was represented by the threshold cycle of amplification (CT), which was inversely correlated to the amount of target RNA/first-strand cDNA being amplified. To normalize for equal amounts of the latter, the transcript levels of the putative housekeeping gene GAPDH were assayed.

For immunoblotting, total cell lysates were prepared. Cells were washed with phosphate-buffered saline pH 7.4, and suspended in lysis buffer (10 mM Tris, pH 7.4, 150 mM, NaCl, 0.1% Triton X-100, 0.5% sodium deoxycholate, and 5 mM EDTA) containing protease inhibitors (Complete tablets; Roche, Ind.). Nuclear extracts were isolated after a cytoplasm protein extraction by incubating the cells with the cytoplasm buffer on ice for 15 min (10 mM HEPES, 10 mM KCl, 0.1 mM EDTA, 0.1 mM EGTA, DTT 1 mM, NP40 0.65%, protease inhibitors, pH 7.4) and centrifugation at 4° C. for 1 min at 10,000 g. The pellet was suspended in the lysis buffer (10 mM Tris, pH 7.4, 150 mM, NaCl, 0.1% Triton X-100, 0.5% sodium deoxycholate, SDS 1.7%, glycerol 5% and 5 mM EDTA) for 30 min and then centrifuged at 4° C. at maximum speed for 20 min. Protein concentrations were measured by the Bradford assay. Equal amounts of protein were subjected to sodium dodecyl sulphate (SDS)-polyacrylamide gels, followed by transfer to polyvinylidene difluoride membranes. Membranes were probed with polyclonal rabbit anti-human survivin (1 μg/mL) (NOVUS), monoclonal mouse anti-human p53 (0.5 μg/mL), polyclonal rabbit anti-human GR (0.5 μg/mL), both from Santa Cruz Biotechnologies, CA; or with mouse anti-human GADPH (Trevigen, Gaithersburg, Md.). Secondary antibodies (GE Healthcare, Chalfont St Giles, United Kingdom) were horseradish peroxidase-conjugated goat anti mouse IgG (1:10,000, v/v) and anti rabbit (1:5000, v/v). Detection was performed by the enhanced chemical luminescence method (Pierce, Rockford, Ill.).

Ciclopirox decreased survivin mRNA and protein expression in wild type HeLa cells as assessed by Q RT-PCR and immunobloting, respectively.

### Example 4

### Leukemia Stem Cells

Ciclopirox was tested for its ability to reduce the viability of TEX and M9-ENL1 cells. TEX and M9-ENL1 cells were derived from lineage-depleted human cord blood cells (Lin-CB) transduced with TLS-ERG or MLL-ENL oncogenes, respectively, and displayed properties similar to leukemia stem cells such as a hierarchal differentiation and marrow repopulation. TEX and M9-ENL1 cells were treated with ciclopirox at a final concentration of 1 or 5 μM. Seventy-two hours after incubation, cell viability was measured by the Alamar Blue assay.

Ciclopirox was found to be able to reduce the viability of TEX and M9-ENL1 cells by at least 75%, with LD₅₀ of 1.5 and 2.5 μM, respectively.

### Example 5

### Mouse Xenograft Models

Mouse xenograft models were used to evaluate the in vivo anticancer activity of ciclopirox.

Mouse xenograft models were prepared by injecting MDAY-D2 (MDAY) murine leukemia cells (5×10⁵) intraperitoneally or subcutaneously into NOD/SCID mice (Ontario Cancer Institute, Toronto, ON); or by inoculating subcutaneously in the flanks of sublethally irradiated NOD-SCID mice (3.5 Gy) with OCI-AML2 (2×10⁶), K562 cells (2×10⁶), MDAY-D2, or U937 leukemia cells.

Compound treatment was initiated when tumors reached volumes of 200 mm³ at which time mice were randomized to receive 25 mg/kg/day of ciclopirox (treated group) or buffer control (untreated group) byoral gavage for 5 to 7 days. Caliper measurements were performed twice weekly to estimate tumor volume (tumor length×width²×0.5236) (Pham et al., Mol. Cancer. Ther. 2004, 3, 1239-1248) and differences compared between treated and untreated groups. Eight (MDA Y-D2), sixteen (OCI-AML2), or thirty (K562) days after injection of cells, mice were sacrificed, and the volume and weight of the tumors were measured. Compared to buffer control, oral ciclopirox decreased tumor weight and volume in all 3 models. No gross organ toxicity or loss of body weight was noted after ciclopirox treatment.

Alternatively, primary AML cells were injected intrafemorally into the right femur of sublethally irradiated nude/NOD/SCID female mice. Four weeks after injection, mice were treated with ciclopirox (20 mg/kg/day) 5/7 days for 4 weeks. At the end of the experiment, the mice were sacrificed, and cells were flushed from the femurs. Engraftment of human cells into the marrow was assessed by enumerating the percentage of human CD45 cells using APC-Cy7-anti-CD45 and flow cytometry. Engrafted cells were confirmed to be leukemic in origin by the presence of human CD33 and lack of CD19. Compared to mice treated with buffer alone, treatment with ciclopirox significantly decreased the engraftment of primary AML cells without gross organ toxicity or loss of body weight.

### Example 6

### Pharmacokinetics of Ciclopirox

Pharmacokinetic parameters of ciclopirox were determined in rats and dogs using [¹⁴C]-ciclopirox olamine. Ciclopirox was administered orally to the rats and dogs. In the rats, ciclopirox was administered orally at a dosing level of 1 mg/kg, and a Cₘₐₓ of 0.083-0.17 μg/mL was observed at 0.25 hrs postdose with a t_{1/2} of 6.8-7.6 hrs. In the dogs, ciclopirox was administered orally at a dosing level of 15 mg/kg/day, and a Cₘₐₓ of 2-7.5 μg/mL was observed within 1.5-2 hours. In a 90 day oral repeat-dosing study in dogs, the mean Cₘₐₓ was 3.9 μg/mL, following a ciclopirox olamine dose of 10 mg/kg/day.

presence of human CD33 and lack of CD19.

### Example 6

### Cell Proliferation Assay and the Determination of IC₅₀

### Adherent Cells

On day 0, cells are seeded at 20,000 cells per well in 100 μL of media into individual wells of a 96-well tissue culture plate. The next day, compounds are diluted in 100 μL of media for a total of 200 μL. Each concentration of the compounds is prepared at 1000× in DMSO (e.g., for a final concentration of 20 μM in the assay, the compounds are prepared at 20 mM in 100% DMSO). The compounds are then diluted 1:500 in media and added in the amount of 100 μl, to each well for a final concentration of 1:1000 with 0.1% DMSO. Each concentration of the compounds is tested in triplicate. Cells are incubated at 37° C. with 5% CO₂. After 72 hours, 20 μl, of CellTiter 96 Aqueous One Solution Cell Proliferation Assay (Promega) is added to each well. Cells are placed back in the incubator, and the absorbance at 490 nm is read after 2-3 hours. The concentration of the compounds that decreases the number of metabolically active cells by 50% is determined and reported as the IC₅₀. “Percent Viability” is determined by subtracting the average background value (media only) and expressed as a ratio to the average value obtained from cells treated with only DMSO.

### Suspension Cells

Assays with suspension cells are similar except that 40,000-60,000 cells are added to each well and compounds are added immediately after cell plating.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A method of treating a drug-resistant hematologic malignancy in a subject, which comprises administering to the subject a therapeutically effective amount of 6-cyclohexyl-1-hydroxy-4-methyl-2(1H)-pyridone, or a pharmaceutical salt or solvate thereof; with the proviso that the drug-resistant leukemia is not imatinib-resistant CML.

2. The method of claim 1, wherein the drug-resistant hematologic malignancy is drug-resistant leukemia.

3. The method of claim 2, wherein the drug-resistant leukemia is drug-resistant acute leukemia.

4. The method of claim 3, wherein the drug-resistant leukemia is drug-resistant ALL.

5. The method of claim 3, wherein the drug-resistant leukemia is drug-resistant AML.

6. The method of claim 2, wherein the drug-resistant leukemia is drug-resistant chronic leukemia, with the proviso that the drug-resistant leukemia is not imatinib-resistant CML.

7. The method of claim 6, wherein the drug-resistant leukemia is drug-resistant CLL.

8. The method of claim 6, wherein the drug-resistant leukemia is drug-resistant CML, with the proviso that the drug-resistant leukemia is not imatinib-resistant CML.

9. The method of claim 2, wherein the drug-resistant leukemia is resistant to a Bcr-Abl kinase inhibitor.

10. The method of claim 9, wherein the Bcr-Abl kinase inhibitor is imatinib, dasatinib, nilatinib, or bosutinib.

11. The method of claim 2, wherein the drug-resistant leukemia is resistant to cytarabine or vincristine.

12. The method of claim 2, wherein the drug-resistant leukemia is Philadelphia positive.

13. The method of claim 2, wherein the drug-resistant leukemia is relapsed or refractory.

14. The method of claim 1, wherein 6-cyclohexyl-1-hydroxy-4-methyl-2(1H)-pyridone, or a pharmaceutical salt or solvate thereof is administered orally.

15. The method of claim 14, wherein the therapeutically effective amount is about 2, about 5, about 10, about 15, or about 20 mg/kg/day.

16. The method of claim 1, wherein 6-cyclohexyl-1-hydroxy-4-methyl-2(1H)-pyridone, or a pharmaceutical salt or solvate thereof is administered parenterally.

17. The method of claim 16, wherein 6-cyclohexyl-1-hydroxy-4-methyl-2(1H)-pyridone, or a pharmaceutical salt or solvate thereof is administered intravenously.

18. The method of claim 16, wherein the therapeutically effective amount is ranging from 0.001 to about 20 mg/kg/day.

19. The method of claim 18, wherein the therapeutically effective amount is ranging from 0.05 to about 0.95 mg/kg/day.

20. The method of claim 16, wherein 6-cyclohexyl-1-hydroxy-4-methyl-2(1H)-pyridone, or a pharmaceutical salt or solvate thereof, is administered intramuscularly.

21. The method of claim 16, wherein 6-cyclohexyl-1-hydroxy-4-methyl-2(1H)-pyridone, or a pharmaceutical salt or solvate thereof, is administered subcutaneously.

22. The method of claim 1, wherein 6-cyclohexyl-1-hydroxy-4-methyl-2(1H)-pyridone, or a pharmaceutical salt or solvate thereof, is administered topically.

23. The method of claim 1, wherein 6-cyclohexyl-1-hydroxy-4-methyl-2(1H)-pyridone, or a pharmaceutical salt or solvate thereof, is administered once per day, twice per day, or three times per day for about 1 to about 26 six weeks.

24. The method of claim 23, wherein the administration is followed by a rest period, during which 6-cyclohexyl-1-hydroxy-4-methyl-2(1H)-pyridone, or a pharmaceutical salt or solvate thereof, is not administered.

25. The method of claim 24, wherein the rest period is one, two, three, four, five, six, or seven days; two, three, or four weeks.

26. The method of claim 2, wherein the subject has not been treated with anticancer therapy for the drug resistant leukemia prior to the administration of 6-cyclohexyl-1-hydroxy-4-methyl-2(1H)-pyridone, or a pharmaceutical salt or solvate thereof.

27. The method of claim 2, wherein the subject has been treated with anticancer therapy for the drug resistant leukemia prior to the administration of 6-cyclohexyl-1-hydroxy-4-methyl-2(1H)-pyridone, or a pharmaceutical salt or solvate thereof.

28. A method for treating leukemia in a subject, which comprises oral administration of 6-cyclohexyl-1-hydroxy-4-methyl-2(1H)-pyridone, or a pharmaceutical salt or solvate thereof, to the subject at a dosage of about 2, about 5, about 10, about 15, or about 20 mg/kg/day.

29. A method for treating leukemia in a subject, which comprises intravenous administration of 6-cyclohexyl-1-hydroxy-4-methyl-2(1H)-pyridone, or a pharmaceutical salt or solvate thereof, to the subject at a dosage ranging from about 0.001 to about 20 mg/kg/day.

30. The method of claim 29, wherein the dosage is ranging from about 0.05 to 0.95 mg/kg/day.

31. A method for treating leukemia in a subject, which comprises administration of 6-cyclohexyl-1-hydroxy-4-methyl-2(1H)-pyridone, or a pharmaceutical salt or solvate thereof, in an amount sufficient to provide a plasma concentration of 6-cyclohexyl-1-hydroxy-4-methyl-2(1H)-pyridone at steady state ranging from about 1 to about 20 μM.

32. A method for treating leukemia in a subject, which comprises administration of 6-cyclohexyl-1-hydroxy-4-methyl-2(1H)-pyridone, or a pharmaceutical salt or solvate thereof, in an amount sufficient to provide a maximum plasma concentration of 6-cyclohexyl-1-hydroxy-4-methyl-2(1H)-pyridone ranging from about 0.1 to about 50 μM.

33. A method for treating leukemia in a subject, which comprises administration of 6-cyclohexyl-1-hydroxy-4-methyl-2(1H)-pyridone, or a pharmaceutical salt or solvate thereof, in an amount sufficient to provide a maximum plasma concentration of 6-cyclohexyl-1-hydroxy-4-methyl-2(1H)-pyridone ranging from about 1 to about 50 μM when two or more doses of 6-cyclohexyl-1-hydroxy-4-methyl-2(1H)-pyridone are administered.

34. The method of claim 28, The method of claim 28, wherein the leukemia is acute leukemia.

35. The method of claim 34, wherein the acute leukemia is ALL.

36. The method of claim 34, wherein the acute leukemia is AML.

37. The method of claim 28, wherein the leukemia is chronic leukemia.

38. The method of claim 37, wherein the chronic leukemia is CLL.

39. The method of claim 37, wherein the acute leukemia is CML.

40. The method of claim 28, wherein the leukemia is drug-resistant.

41. The method of claim 40, wherein the leukemia is resistant to a Bcr-Abl kinase inhibitor.

42. The method of claim 41, wherein the Bcr-Abl kinase inhibitor is imatinib, dasatinib, nilatinib, or bosutinib.

43. The method of claim 28, wherein the leukemia is resistant to cytarabine or vincristine.

44. The method of claim 28, the leukemia is Philadelphia positive.

45. The method of claim 28, the leukemia is relapsed or refractory.

46. A method of inhibiting the growth of a leukemia stem cell, comprising the step of contacting the cell with 6-cyclohexyl-1-hydroxy-4-methyl-2(1H)-pyridone, or a pharmaceutical salt or solvate thereof.

47. The method of claim 46, wherein the leukemia stem cell is an acute leukemia stem cell.

48. The method of claim 47, wherein the leukemia stem cell is an ALL or AML stem cell.

49. The method of claim 46, wherein the leukemia stem cell is a chronic leukemia stem cell.

50. The method of claim 49, wherein the leukemia stem cell is a CLL or CML stem cell.

51. The method of claim 46, wherein the leukemia stem cell is drug resistant.

52. The method of claim 51, wherein the leukemia stem cell is resistant to a Bcr-Abl kinase inhibitor.

53. The method of claim 52, wherein the Bcr-Abl kinase inhibitor is imatinib, dasatinib, nilatinib, or bosutinib.

54. The method of claim 51, wherein the leukemia stem cell is cytarabine-resistant or vincristine-resistant.

55. The method of claim 46, wherein the leukemia stem cell is Philadelphia positive.


SEQUENCE LISTING (6 sequences)

SEQ ID NO: 1 - DNA, 35 bp, Artificial sequence
  Forward primer
ggcgagctcactttttctgtcacctccgtggtccg

SEQ ID NO: 2 - DNA, 28 bp, Artificial Sequence
  Reverse primer
gggttcgaaacggcggcggcggtggaga

SEQ ID NO: 3 - DNA, 20 bp, Artificial Sequence
  survivin forward primer
ttttcatcgtcgtccctagc

SEQ ID NO: 4 - DNA, 20 bp, Artificial Sequence
  survivin reverse primer
cgactcagatgtggcagaaa

SEQ ID NO: 5 - DNA, 19 bp, Artificial Sequence
  GAPDH forward primer
gaaggtgaaggtcggagtc

SEQ ID NO: 6 - DNA, 20 bp, Artificial Sequence
  GAPDH reverse  primer
gaagatggtgatgggatttc